(19) United States
(12) Patent Application Publication
(10) Pub. No.: US 2016/0121093 A1
(43) Pub. Date: May 5, 2016

(54) WIRELESS TATTOOING APPARATUS

(71) Applicant: MEI-CHI-NA HSINYEN CO., LTD., Taipei (TW)

(72) Inventor: CING-RONG FAN, TAIPEI (TW)

(21) Appl. No.: 14/928,578

(22) Filed: Oct. 30, 2015

(30) Foreign Application Priority Data
Oct. 31, 2014 (TW) 103219300

(51) Int. Cl. A61M 37/00; H04W 4/00

(52) U.S. Cl. CPC A61M 37/0076; H04W 4/008

(57) ABSTRACT

A wireless tattooing apparatus to be wirelessly controlled by a wireless remote-control device is disclosed. The wireless tattooing apparatus may include a main body having a wireless control module, a driving mechanism, with the driving mechanism axially connected with a needle extending outside of the main body, and a motor coupled to the driving mechanism. The motor is controlled and driven by the wireless control module to actuate the driving mechanism, which in turn drives the needle to displace back and forth with respect to the main body. Operations of the wireless tattooing apparatus may be controlled and configured by the wireless remote-control device.

### CROSS-REFERENCE TO RELATED APPLICATIONS

This non-provisional application claims priority under 35 U.S.C. §119(a) on Patent Application No. 103219300 filed in Taiwan, R.O.C. on Oct. 31, 2014, the entire contents of which are hereby incorporated by reference.

### BACKGROUND

1. Technical Field

The present disclosure relates to a wireless tattooing apparatus, in particular, to a wireless tattooing apparatus controllable and configurable by a wireless remote-control device.

2. Description of Related Art

Tattooing technology is actually invasive skin coloration. As human skin may be typically divided into epidermis and dermis with epidermis cuticle sandwiched between the epidermis and dermis. The epidermis cuticle is composed of squamous cells while the epidermis contains no blood vessels but is with numerous tiny nerve endings and the epidermis cuticle is basically translucent.

The tattooing technology aims to plant stable color pigment in the skin tissue, and due to the thinness and translucence of the skin such color pigment through the epidermis could conceal blemishes and/or modify the appearance. However, the performance of the tattooing may be associated with certain risk and thus specialized equipments are necessary.

Generally, tattooing equipments are driven by electrical motors, which are connected to power cords/plugs connecting to city power. Anyone performing the tattooing needs to concentrate and the presence of the power cords or power plugs transmitting the power from the city power to the equipments often distracts the people performing the tattooing and even limits the displacement and the movement angle of the equipments.

### SUMMARY OF THE DISCLOSURE

The present disclosure is to provide a wireless tattooing apparatus controllable and configurable by a wireless remote-control device, to minimize the limitation and interference associated with the power cord or power plug on the displacement and the movement angle of the traditional tattooing apparatus.

The wireless tattooing apparatus may include a main body having a wireless control module, a driving mechanism, with the driving mechanism axially connected with a needle extending outside of the main body, and a motor coupled to the driving mechanism. The motor is controlled and driven by the wireless control module to actuate the driving mechanism, which in turn drives the needle to displace back and forth with respect to the main body.

As the wireless tattooing apparatus of the present disclosure may no longer subject to the limitation or interference associated with the power cord or power plug, the user may concentrate more on the tattooing and the movement of the wireless tattooing apparatus may be more smooth and more accurate.

For further understanding of the present disclosure, reference is made to the following detailed description illustrating the embodiments and examples of the present disclosure. The description is only for illustrating the present disclosure, not for limiting the scope of the claim.

### BRIEF DESCRIPTION OF THE DRAWINGS

The drawings included herein provide further understanding of the present disclosure. A brief introduction of the drawings is as follows:

FIG. 1 is a cross-sectional view of a wireless tattooing apparatus according to one embodiment of the present disclosure;

FIG. 2 is a simplified block diagram of a wireless tattooing apparatus according to one embodiment of the present disclosure; and

FIG. 3 is a simplified block diagram showing remote control of the wireless tattooing apparatus by a wireless remote-control device according to one embodiment of the present disclosure.

### DESCRIPTION OF THE EXEMPLARY EMBODIMENTS

The aforementioned and other technical contents, features, and efficacies will be shown in the following detail descriptions of a preferred embodiment corresponding with the reference Figures.

A wireless tattooing apparatus according to the present disclosure may inject the ink into the skin of the human being. For the illustration purpose, the wireless tattooing apparatus may be pen-shaped throughout the present disclosure. However, it is well known in the pertinent art that the shape of the tattooing device may not be limited as the result.

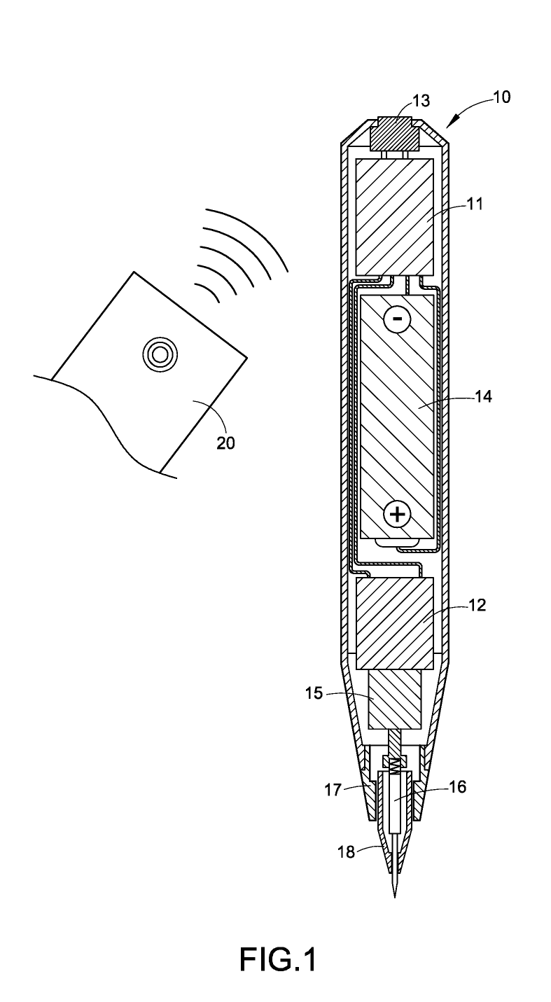

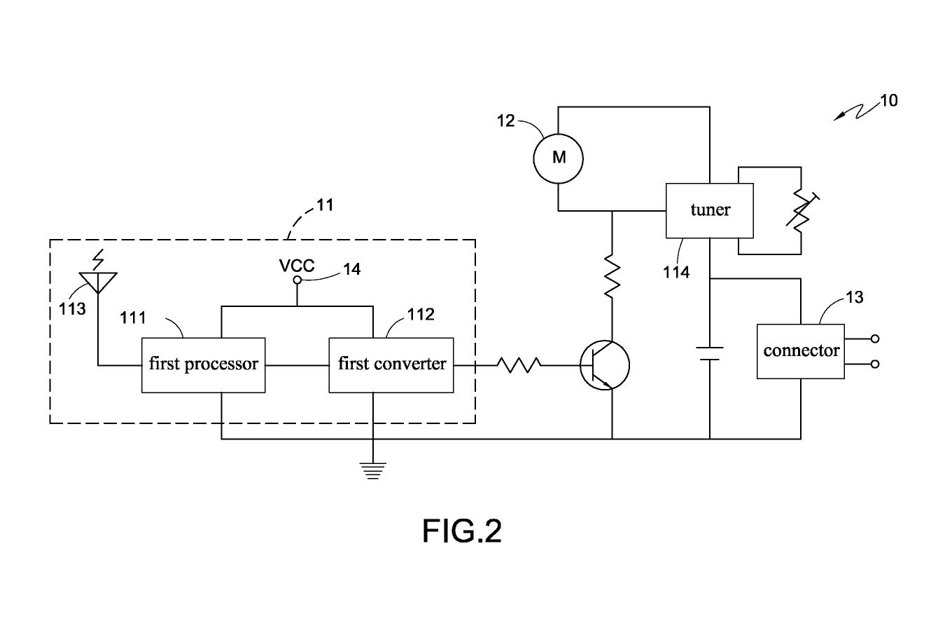

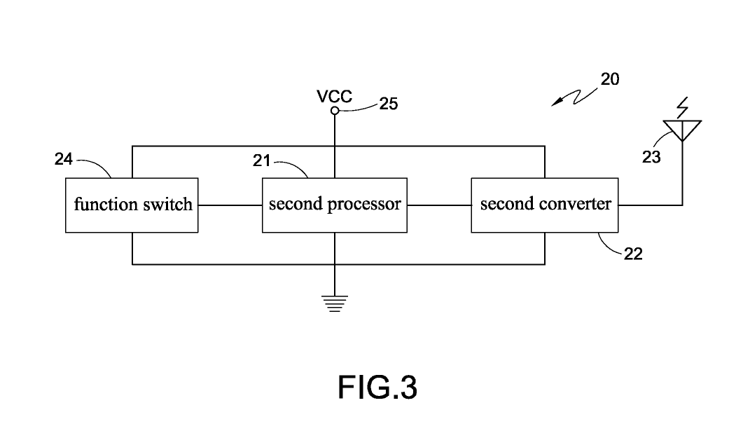

Please refer to FIGS. 1-3 showing the wireless tattooing device wirelessly controllable by a wireless remote-control device 20. The wireless tattooing apparatus may include a main body 10. In one implementation, the main body 10 may be implemented as a pen allowing for the user to hold the main body 10 (or the wireless tattooing apparatus) when performing the corresponding tattooing task. The main body 10 may include a wireless control module 11, a motor 12, a connector 13, and a battery 14 therein. The main body 10 may also include a driving mechanism 15 axially connected to a needle 16. The needle 16 may extend outside the main body 10. The motor 12 may be connected to the driving mechanism 15 and may therefore cause the driving mechanism 15 to displace the needle 16 back and forth, allowing for the ink stored within the needle 16 to be injected into the skin of the human being when the tattooing is underway. Moreover, the main body 10 may be with a holder 17 corresponding to the position of the needle 16. With the holder 17, the user performing the tattooing may hold on to the main body 10 with his/her fingers. A needle cover 18 may be positioned to surround the needle 16. The needle cover 18 may be integrated with the holder 17. Design of the driving mechanism 15, the needle 16, the holder 17, and the needle cover 18 may be well known in the pertinent art and therefore could be adaptive to accommodate the needs.

The wireless control module 10 placed within the main body 10 may include a first processor 111, a first converter 112, a first antenna 113, and a tuner 114. The first antenna 113 may be used to receive a functional signal transmitted by the wireless remote-control device 20, while the first processor 111 may transmit the functional signal to the first converter 112, which may in turn convert the functional signal to a control signal. The tuner 114 may operate on basis of the control signal from the first converter 112.

The first antenna 113 may be electrically coupled to the first processor 111 for receiving the functional signal transmitted from the wireless remote-control device 20. Such transmission may be through Bluetooth, radio frequency (RF), wireless networking, or infrared transmission communication protocol.

The motor 12 may be placed within the main body 10 and electrically connected to the wireless control module 11. Specifically, the motor 12 may be therefore electrically connected to the tuner 114, which may control the operation of the motor 12 such as the rotating speed and operational time period.

The connector 13 may be placed within the main body 10 and electrically connected to the wireless control module 11. Specifically, one end of the main body 10 away from the location of the needle 16 may be where the connector 13 could be disposed. The connector 13 may be further electrically connected to the first processor 111 of the wireless control module 11.

The battery 14 may be placed within the main body 10 and electrically connected to the wireless control module 11. Specifically, the battery 14 may be electrically connected to the first processor 111 and the first converter 112. In one implementation, the battery 14 may be a recharge-able battery. The connector 13 may be electrically connected to an external charger (not shown) for charging the battery 14 when in operation. The battery 14 may thereafter provide the electricity to the wireless control module 11, which may further distribute the same to the motor 12.

When the wireless tattooing apparatus of the present disclosure is in use, the wireless remote-control device 20 may transmit the functional signal to the wireless tattooing apparatus wirelessly. In order to serve this purpose, the wireless remote-control device 20 may include a second processor 21, a second converter 22, a second antenna 23, a function switch 24, and a power unit 25. The second processor 21 may be electrically connected to the second converter 22 and the second antenna 23. The second converter 22 may be electrically connected to the function switch 24. The function switch 24 may be disposed outside the wireless remote-control device 20. The number of the function switches may vary from case to case. In one implementation, the number of the function switches is two for accelerating and decelerating. The functionality of such function switches may vary from case to case as well. The function switches when activated may deliver their corresponding functional signals.

The power unit 25 may be used to power the second processor 21, the second converter 22, and the function switch 24. When the function switch 24 is activated, the second processor 21 may transmit the corresponding functional signal to the second converter 22. The second antenna 23 may thereafter transmit the functional signal to the first antenna 113 of the wireless control module 11.

For example, the user may press the function switch 24 of the wireless remote-control device 20 to generate one “accelerating” functional signal. Such functional signal may be transmitted to the wireless tattooing apparatus in a wireless fashion. When the first antenna 113 receives the functional signal, the first antenna 113 may transmit the functional signal to the first processor 111 of the wireless control module 11. The first processor 111 upon the receipt of the functional signal may transmit the same functional signal to the first converter 112, which may be used to convert the received functional signal to the corresponding control signal for the tuner 114. The rotating speed of the motor 12 may be accelerated as the result.

Thus, at the time the wireless tattooing apparatus operates environmental factors affecting the operation of the wireless tattooing apparatus may be minimized. No limitation or interference associated with the power cord or power plug may be present when the tattooing using the wireless tattooing apparatus is performed. The wireless remote-control device 20 may be within the reach of the fingers of the user, allowing for the operation of the wireless tattooing apparatus to be controlled by the wireless remote-control device.

As the wireless tattooing apparatus of the present disclosure may no longer subject to the limitation or interference associated with the power cord or power plug, the user may concentrate more on the tattooing and the movement of the wireless tattooing apparatus may be more smooth and more accurate.

Some modifications of these examples, as well as other possibilities will, on reading or having read this description, or having comprehended these examples, will occur to those skilled in the art. Such modifications and variations are comprehended within this disclosure as described here and claimed below. The description above illustrates only a relative few specific embodiments and examples of the present disclosure. The present disclosure, indeed, does include various modifications and variations made to the structures and operations described herein, which still fall within the scope of the present disclosure as defined in the following claims.

What is claimed is:

1. A wireless tattooing apparatus controlled by a wireless remote-control device, comprising a main body having a wireless control module placed within the main body, a driving mechanism placed within the main body, with the driving mechanism axially connected with a needle extending outside of the main body, and a motor placed within the main body and coupled to the driving mechanism, wherein the motor is controlled and driven by the wireless control module to actuate the driving mechanism, which in turn drives the needle to displace back and forth with respect to the main body.

2. The wireless tattooing apparatus according to claim 1, wherein the main body further comprises a holder at one end of the main body and positioned corresponding to the location of the needle and a needle cover integrated with the holder and surrounding the needle.

3. The wireless tattooing device according to claim 1, wherein the wireless control module further comprises a first processor, a first converter, a first antenna for receiving a functional signal transmitted by the wireless remote-control device, and a tuner, wherein the first processor transmits the functional signal to the first converter, wherein the first converter converts the functional signal to a corresponding control signal for the tuner, and wherein the tuner controls the motor on basis of the control signal.

4. The wireless tattooing device according to claim 1, further comprising a connector placed within the main body and electrically connected to the wireless control module.

5. The wireless tattooing device according to claim 1, further comprising a battery placed within the main body and electrically connected to the wireless control module.
